(19) United States
(12) Patent Application Publication
(10) Pub. No.: US 2026/0269048 A1
(43) Pub. Date: Sep. 10, 2026

(54) GRAPH BASED MULTIMODAL SYSTEM FOR GENERATIVE MODEL DRIVEN THERAPEUTIC INTERVENTION

(71) Applicant: WONDER SCIENCES, INC., Marina del Rey, CA (US)

(72) Inventor: Ryan R. Magnussen, Marina del Rey, CA (US)

(21) Appl. No.: 19/556,726

(22) Filed: Mar. 4, 2026

Related U.S. Application Data
(60) Provisional application No. 63/768,822, filed on Mar. 7, 2025.

(51) Int. Cl. G16H 20/70

(52) U.S. Cl. CPC G16H 20/70

(57) ABSTRACT

A system and method provide continuous, data-driven support for mental health treatment by maintaining a persistent state graph that records patient progress across therapy sessions. The system collects multimodal data such as text, audio, video, physiological, and interaction signals to analyze behavioral and emotional patterns. Using these inputs, the system employs a generative language model to propose therapy interventions that are contextually appropriate for the patient’s current psychological state. After intervention, the system monitors new data to measure the patient’s response to the therapy and updates the state graph with relevant session outcomes and relationships among historical, dynamic, and intervention variables. The accumulated information supports both patient-facing and clinician-facing outputs, enabling real-time monitoring, tailored guidance, and longitudinal analysis of progress, engagement, and risk trajectories within a governed digital mental healthcare framework.

### CROSS-REFERENCE TO RELATED APPLICATIONS

The present application claims the benefit of and priority to U.S. Provisional Application No. 63/768,822 filed on Mar. 7, 2025, which is incorporated by reference.

### BACKGROUND

Mental health disorders, including major depressive disorder, anxiety disorders, post-traumatic stress disorder (PTSD), trauma-related disorders, and substance use disorders, represent a leading cause of disability worldwide. These conditions require ongoing assessment, risk monitoring, longitudinal outcome tracking, and structured intervention planning. In regulated healthcare environments, digital systems intended to support mental health treatment must operate with traceability, auditability, safety constraints, and clinician oversight.

Conventional digital therapy systems operate within session-specific context windows that discard patient state information between interactions. Such systems are episodic and resource-constrained and typically consist of discrete therapy sessions separated by extended intervals. This architectural limitation prevents longitudinal tracking of symptom trajectories, intervention-response patterns, and cumulative therapeutic progress. Between sessions, limited structured monitoring of symptom trajectories, relapse risk, autonomic dysregulation, or engagement decay is available. Clinicians frequently rely on retrospective patient recall and manually generated documentation, which may not capture longitudinal patterns or subtle state transitions relevant to patient safety and treatment optimization. Increasing regulatory scrutiny, documentation requirements, and risk management obligations further heighten the need for systems that provide structured, interpretable, and auditable clinical support. Existing digital health systems and mental health chat-bots are not architected as regulated clinical infrastructures. Existing conversational AI systems commonly operate on short-context language models and do not maintain information about a patient's progress across sessions. Additionally, conventional digital therapy systems perceive only those inputs the patient readily provides through a chat interface. The systems also frequently rely on rule-based safety filters without adequate risk assessments.

Further, existing platforms rarely unify patient-facing digital support, clinician-facing dashboards, longitudinal risk modeling, and accredited clinician training workflows within a single governed architecture. As a result, many systems are not readily aligned with certification requirements related to explainability, safety mode control, monitoring, competency, and regulatory documentation.

### SUMMARY

A disclosed system and method addresses the limitations of conventional episodic therapy models by maintaining a persistent state graph across multiple patient therapy sessions. During a session, the system receives multimodal inputs from a patient including text, audio, video, physiological signals, or interaction timing data, and extracts state features from these inputs to provide comprehensive patient monitoring beyond what clinicians can capture through retrospective recall and manual documentation. The system prompts a generative language model with both the extracted state features and a representation of the state graph to propose candidate therapeutic interventions. The proposed interventions can be performed through patient-facing outputs. The system observes changes in the multimodal inputs of the patient following the interventions and updates the state graph based on these observed changes for use in subsequent therapy sessions. The state graph can be saved in an encrypted, version-controlled representation.

The state graph encodes how psychological variables, interventions, and outcomes relate to each other through typed edges with confidence weights and temporal metadata. This graph structure enables a generative language model to reason over longitudinal patient context in a manner that is both computationally tractable and clinically interpretable, producing candidate interventions that are grounded in the patient’s full therapeutic history rather than limited to the current session’s input.

The approach provides technical advantages by transforming episodic therapy delivery into a continuous, data-driven system that maintains longitudinal patient state information across sessions. The persistent state graph enables structured monitoring of symptom trajectories, relapse risk, and engagement patterns that are typically unavailable between conventional therapy sessions by integrating multimodal behavioral analytics with cross-session continuity. The system also provides interpretable information about how generative language model outputs integrate with the system. These transparent and traceable outputs, stored in the state graph address the regulatory scrutiny and documentation requirements of modern mental healthcare delivery. The system supports both patient-facing therapeutic interventions and clinician-facing augmentation through longitudinal dashboards and structured documentation artifacts.

### BRIEF DESCRIPTION OF THE DRAWINGS

FIG. 1 is a block diagram illustrating an example system environment for an analytics system in accordance with one or more embodiments.

FIG. 2 illustrates an example system architecture of the analytics system 140, in accordance with one or more embodiments.

FIG. 3 illustrates an example Longitudinal Clinical Memory Graph (LCMG) 300 before a therapeutic intervention.

FIG. 4 depicts an updated example state graph of the example patient after a therapy intervention in accordance with an example embodiment.

FIG. 5 is a flowchart depicting a process for using a patient LCMG to recommend a therapeutic intervention and updating the LCMG based on the outcome of the intervention, in accordance with an example embodiment.

FIG. 6 illustrates a structure of an example neural network, in accordance with some embodiments.

FIG. 7 is a conceptual diagram of functional blocks of a transformer-based neural network model, in accordance with some embodiments.

The figures depict various embodiments for purposes of illustration only. One skilled in the art will readily recognize from the following discussion that alternative embodiments of the structures and methods illustrated herein may be employed without departing from the principles described here.

### DETAILED DESCRIPTION

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### System and Networking Environment

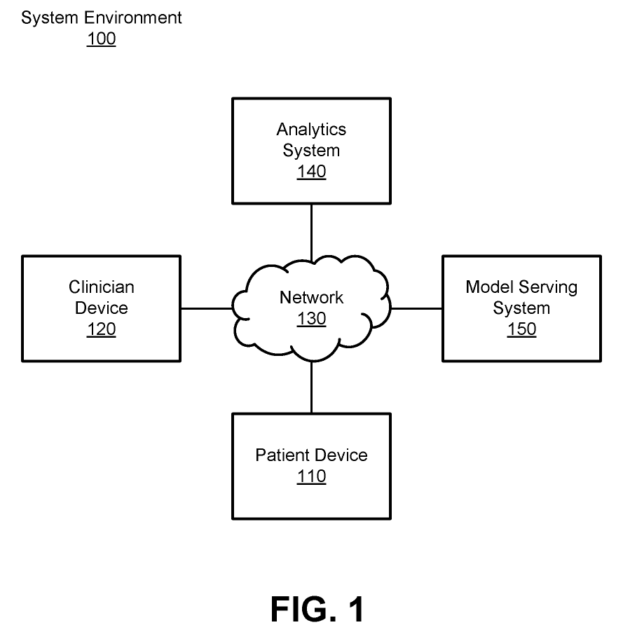

FIG. 1 is a block diagram illustrating an example system environment for an analytics system in accordance with one or more embodiments. The system environment illustrated in FIG. 1 includes a patient device 110, a clinician device 120, a network 130, an analytics system 140, and a model serving system 150. Alternative embodiments may include more, fewer, or different components from those illustrated in FIG. 1, and the functionality of each component may be divided among the components differently from the description below. Additionally, each component may perform its respective functionalities in response to a request from a human (e.g., a patient or clinician) or automatically without human intervention.

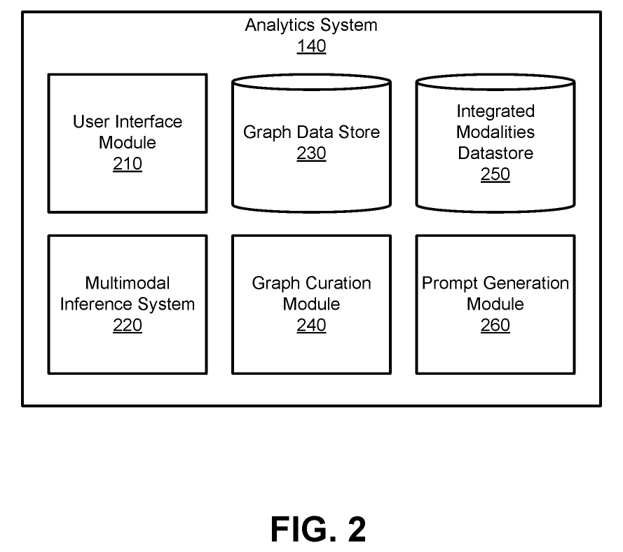

In operation, the analytics system 140 maintains and updates a longitudinal state graph that records psychological state variables, therapy events, and measured outcomes across successive sessions. This state graph provides the structural foundation for reasoning over historical and real-time data, enabling both patient-facing and clinician-facing components to generate context-aware therapeutic insights and recommendations. The architecture of the analytics system 140 and its associated functionality is further described in FIGS. 2–5.

In some embodiments, the state graph may comprise a network of interconnected nodes and edges that collectively model the evolving psychological and therapeutic profile of a patient. Each node represents a clinically relevant element such as a psychological state variable, intervention event, or outcome measure, and may include associated metadata including timestamps, confidence scores, or provenance indicators. The edges of the state graph define the relationships among nodes which may include temporal connections linking successive sessions, causal or associative links denoting influence or correlation between states, and weighted contributions that quantify the relative strength or significance of such relationships. Collectively, the nodes and edges form a structured, interpretable representation that enables the system to reason about longitudinal patterns, therapeutic responses, and cross-session dependencies in a traceable and clinically governed manner.

While one patient device 110 and one clinician device 120 are illustrated in FIG. 1, any number of patients and clinicians may interact with the analytics system 140. Accordingly, there may be multiple patient devices 110, clinician devices 120, analytics systems 140, or model serving systems 150 deployed in different configurations. As used herein, patients and clinicians may be generically referred to as “users” of the analytics system 140. Devices that interact with the analytics system 140, including patient devices 110 and clinician devices 120, may also be generally referred to herein as “client devices” or “user devices.”

The patient device 110 is a client device through which a patient or other user can interact with the analytics system 140 over the network 130. The patient device 110 may be any personal or mobile computing device, such as a smartphone, a tablet, a laptop computer, or a desktop computer. In some embodiments, the patient device bincludes or is connected to sensors or input systems (e.g., microphone, camera, motion sensors, or physiological sensors) to capture multimodal signals such as text, audio, video, or biometric data during therapeutic interactions. In addition, the patient device 110 may include or be coupled to output components such as a display, speaker, vibration motor, or haptic feedback system that render visual, auditory, or tactile therapeutic outputs to support guided exercises, mindfulness routines, or stabilizing interventions. In some embodiments, the patient device 110 may execute a client application that provides a therapeutic interface and uses an application programming interface (API) to communicate securely with the analytics system 140.

Through the therapeutic interface of the patient device 110, the patient can participate in guided therapeutic sessions, complete exercises or journaling prompts, and receive real-time stabilizing or skills-training content generated by the analytics system 140. The interface presented on the patient device 110 may include text or voice-based therapeutic exchanges, scheduled coursework, or interactive tools for self-regulation, all governed by the analytics system 140 under applicable clinical safety protocols.

In some embodiments, the patient device 110 presents patient-facing outputs generated by the analytics system 140 to deliver guided therapeutic content and exercises between or during sessions. These outputs may include cognitive restructuring prompts aimed at reframing maladaptive thoughts; skills-coaching protocols that support communication, mindfulness, or emotional regulation; and stabilization exercises to reduce acute distress or autonomic dysregulation. The interface may further provide structured between-session coursework or values-reflection modules for deepening insight and adherence to therapy objectives. Additional features may include sleep and circadian rhythm supports, relational skill exercises, habit-formation scaffolding, and relapse-prevention planning tools integrated into the patient’s longitudinal care plan. The patient device 110 may adapt presentation timing and intensity based on feedback signals and longitudinal graph-derived indicators, ensuring personalized, safety-aligned digital interventions for continuous therapeutic engagement.

The clinician device 120 is a client device through which a licensed clinician interacts with the analytics system 140 over the network 130. The clinician device 120 may be any workstation, mobile device, or tablet configured to view patient-specific data, longitudinal state graphs, predicted risk trajectories, and suggested intervention options generated by the analytics system 140. In some embodiments, the clinician device 120 may provide dashboards displaying therapeutic progress metrics, risk alerts, and documentation templates, enabling clinician oversight and plan adjustment. The clinician device 120 communicates with the analytics system 140 through secure APIs and may send annotations or validation inputs that update clinical records within the analytics system 140.

In some embodiments, the clinician device 120 provides advanced clinician‑facing outputs that present longitudinal analytical views of patient progress and relational dynamics to assist in evidence‑based treatment planning. The clinician interface may render interactive dashboards visualizing schema rigidity trends, attachment activation cycles, therapeutic alliance durability metrics, relapse probability curves, dropout hazard estimates, belief‑updating trajectories, or some combination thereof derived from longitudinal analysis of the patient’s state graph. For example, schema rigidity trends may illustrate how firmly a patient’s core cognitive or interpersonal schemas resist modification across sessions and highlight areas where flexible thinking may be limited. Attachment activation cycles may depict recurring temporal patterns of attachment-related behaviors, such as shifts between proximity-seeking and avoidance responses, derived from observed interaction data. Therapeutic alliance durability metrics may quantify the strength, stability, and continuity of the collaborative relationship between clinician and patient, reflecting rapport and treatment engagement signals. Relapse probability curves may estimate the likelihood of symptom resurgence over time based on model-inferred state trajectories and historical outcome measures. Dropout hazard estimates may represent the system’s monitored probability that a patient may disengage or terminate therapy prematurely, guiding proactive retention interventions. Belief-updating trajectories may track directional changes in a patient’s underlying cognitive or emotional belief structures, indicating progress in restructuring maladaptive thought patterns and adaptive insight formation.

The clinician device 120 may further display structured documentation artifacts and care plan update templates that incorporate model‑generated interpretations and clinician annotations. The outputs highlight emerging risk patterns, alliance decay indicators, or cognitive shifts so that licensed clinicians can perform more tailored supervision, validation, and adjustment of treatment plans. Through these clinician‑facing recommendations and visualizations, the analytics system 140 facilitates informed decision‑making, safety monitoring, and coordinated therapeutic interventions across sessions.

The network 130 is a communication network coupling the patient device 110, clinician device 120, analytics system 140, and model serving system 150. The network 130 may include wired or wireless connectivity components and may comprise one or more local area networks (LANs), wide area networks (WANs), or cloud networks. The network 130 may transmit encrypted data using standard communication protocols (e.g., TCP/IP, HTTPS, Bluetooth, or NFC). The network 130 ensures end-to-end secure transport of clinical data consistent with regulated privacy standards.

The analytics system 140 manages and records therapy sessions and progress. In various embodiments, the analytics system may process patient interactions, evaluate therapeutic data, and generate governed outputs for presentation both patients and clinicians. In various embodiments, the analytics system 140 includes processing components including graph storage for encrypted longitudinal clinical memory graphs representing patient state, intervention libraries, therapeutic training data, safety policies, multimodal feature extractors, or some combination thereof. The analytics system 140 may further include prompt generation engines, risk-state estimation modules, and supervisory safety controllers configured to interpret multimodal data received from the patient device 110 and to generate context-appropriate therapeutic or clinician-facing outputs. The analytics system 140 may log inference events and updates in compliance with software-as-a-medical-device (SaMD) requirements and can retrieve locally stored or cached model parameters when executing certain operations.

The model serving system 150 hosts and serves one or more generative and inference machine-learning models used by the platform, such as transformer-based language models or multimodal reasoning engines. The model serving system 150 may perform tasks including natural-language generation, probabilistic belief updating, multimodal feature fusion, and context-aware therapeutic prompt completion. Requests from the analytics system 140 are transmitted to the model serving system 150 through the network 130, and the model serving system 150 returns model-generated outputs or embeddings for further processing. In some embodiments, the analytics system 140 may additionally run locally stored or specialized models alongside or in place of the remote model serving system 150.

The patient device 110, clinician device 120, analytics system 140, and model serving system 150 cooperate to create a closed-loop therapeutic control architecture. Multimodal input and feedback from the patient device 110 are analyzed by the analytics system 140 and the model serving system 150 to derive updated state estimates and recommended interventions. Corresponding clinician-facing insights may be presented via the clinician device 120 for review, documentation, and oversight, ensuring that therapeutic interaction and supervision remain clinically governed and traceable. Alternative embodiments may integrate additional modules (e.g., specialized privacy gateways, data aggregation nodes, or local inference engines) without departing from the scope of the described system environment.

FIG. 2 illustrates an example system architecture of the analytics system 140, in accordance with one or more embodiments. The analytics system 140 includes one or more modules for processing multimodal inputs from patient and clinician devices and generating governed therapeutic and clinician-facing outputs. The analytics system 140 includes a user interface module 210, a multimodal inference system 220, a graph data store 230, a graph curation module 240, an integrated modalities datastore 250, and a prompt generation module 260. Alternative embodiments may include more, fewer, or different components than those illustrated in FIG. 2, and the configuration, interconnection, and division of functionality among the depicted modules may vary.

The user interface module 210 manages interaction interfaces and input collection for patient-facing and clinician-facing applications. The user interface module 210 renders graphical interfaces accessible on client devices such as patient device 110 and clinician device 120, including text windows, voice input controls, sensor integration panels, and visualization dashboards. The user interface module 210 collects multimodal interaction signals such as text, audio, video, timing, and physiological data, and transmits those inputs securely to downstream processing modules within the analytics system 140. In some embodiments, the user interface module 210 also presents reasoning outputs, therapy recommendations, progress summaries, or some combination thereof to the user, optionally including uncertainty indicators or longitudinal trends derived from state modeling.

For example, in some embodiments, the user interface dynamically updates displayed content as the patient interacts with the system in real-time. During a therapeutic exercise, the interface may visualize longitudinal progress indicators that respond to ongoing multimodal input, such as changes in vocal tone, text sentiment, or facial expression, by adjusting progress gauges or highlighting current focus areas derived from the underlying state graph. If the system detects reduced affective volatility or improved coherence in reflective responses, the display may transition to reinforcement messages or present follow-up prompts tailored to the detected stabilization. Conversely, when uncertainty indicators increase, the interface may render supplementary guidance or calming interventions. This adaptive interaction flow enables continuous and personalized feedback for both patient and clinician contexts.

The multimodal inference system 220 processes synchronized multimodal data streams to extract structured features usable for clinical reasoning. The multimodal interface system 220 receives user input data from the user interface module 210 and performs feature extraction across text, audio, video, or physiological channels. The multimodal inference system 220 produces feature representations capturing behavioral and emotional markers such as speech cadence, tone, facial expression changes, gaze variation, timing patterns, or affect volatility. These extracted features are subsequently provided to the graph curation module 240 for updating a patient’s longitudinal state graph and to the prompt generation module 260 for inclusion in therapy prompt construction.

In certain embodiments, the multimodal inference system 220 may implement specialized feature extraction pipelines across visual and textual modalities to enable comprehensive clinical reasoning and support both patient- and clinician-facing outputs. For video signals, the multimodal interface system 220 may employ computer-vision models (e.g., convolutional or transformer-based neural networks) to detect and quantify facial expression changes, gaze variance, and micro-expression dynamics that reflect evolving affective states. For text inputs, the system may apply natural language processing engines, such as contextual semantic parsers or transformer encoders, to analyze word patterns, sentiment polarity, and syntactic constructions indicative of cognitive themes or emotional trends in patient communications. Extracted video and text features may be fused into structured representations and transmitted to downstream modules, including the prompt generation module 260, which constructs context-rich prompts for proposing therapeutic interventions.

In some embodiments, the multimodal inference system 220 may employ a state-estimation model that computes a latent representation of a patient’s current psychological state based on received visual and textual features. A state-estimation model may implement probabilistic inference or deep-learning architectures to derive high-level psycho-state indicators from multimodal signals such as stress level, mood stability, or attention variance. These predicted psycho-state nodes can be linked to a longitudinal state graph maintained in the system memory, to update the historical context used for clinical reasoning. The inferred state representations may then used by the prompt-generation module 260 and other downstream cognitive-support components to tailor therapeutic recommendations, dialogue structure, and visualization outputs to the patient’s real-time psychological state and trajectory.

In some embodiments, the multimodal inference system 220 may additionally include a cross-modal temporal alignment layer that synchronizes extracted features across modalities to a common temporal reference frame. Because text, audio, video, and physiological signals may be sampled at different rates and with different latencies, the alignment layer may resample and interpolate feature vectors to produce time-aligned multimodal frames. The aligned frames are then processed by a model that generates a joint embedding that captures cross-modal correlations, for example, the co-occurrence of increased vocal pitch (audio), reduced gaze stability (video), and negative sentiment shift (text) within a defined temporal window. The fused feature embedding output can serve as a primary input to both the graph curation module 240 and the prompt generation module 260.

The graph data store 230 maintains patient-specific longitudinal state graphs, each represented as an encrypted Longitudinal Clinical Memory Graph (LCMG). An LCMG comprises time-indexed nodes and edges that encode a patient’s psychological and therapeutic history. Nodes may include, for example, trait prior nodes representing stable characteristics (e.g., baseline personality or temperament), dynamic state nodes representing session-dependent variables such as mood, anxiety, or relational status, intervention nodes representing therapies delivered, and outcome nodes capturing measured progress or response variables. Edges record relationships among nodes, such as temporal edges linking session sequences, causal edges linking intervention to outcome, and associative edges connecting related emotional or behavioral states. Each node and edge may include metadata fields such as timestamps, confidence weights, temporal decay factors, uncertainty bounds, provenance labels, clinician validation flags, access-control tags, and cryptographic integrity identifiers. The graph data store 230 thus provides a secure, auditable structure for representing longitudinal continuity and supporting governed therapy recommendations.

In some embodiments, the graph data store 230 may determine node and edge types dynamically during graph construction and subsequent curation by analyzing both the source and context of incoming data. When new multimodal inputs, session records, or clinician annotations are processed, the analytics system 140 classifies each data element according to its semantic role and temporal characteristics. Persistent or personality-linked variables are designated as trait prior nodes, transitory or session-specific measures become dynamic state nodes, discrete therapeutic actions are recorded as intervention nodes, and measured results or longitudinal progress indicators form outcome nodes. Edge types may then be inferred based on the logical and temporal relationships among nodes: sequential session order produces temporal edges, statistically correlated or co-varying attributes produce associative edges, and detected cause-and-effect or intervention-response linkages produce causal edges. The graph curation module 240 employs probabilistic modeling, provenance analysis, and rule mappings defined in the system’s clinical ontology to validate these assignments, ensuring that node and edge designations consistently reflect the psychological meaning, data origin, and therapeutic function represented within the encrypted longitudinal clinical memory graph (LCMG).

The graph curation module 240 updates the longitudinal clinical memory graph in response to new data, therapeutic actions, or session outcomes. When a new therapy session occurs or multimodal changes are detected by the multimodal inference system 220, the graph curation module 240 creates or modifies typed nodes and edges in the patient’s LCMG. As an example, an update may include adding a session outcome node representing the effectiveness of a recent intervention, linking that node to prior dynamic state nodes, recalibrating confidence weights and uncertainty metrics, and recording the new edge relationships representing temporal progression or causal influence. The graph curation module 240 applies temporal decay and recency weighting functions to older nodes, ensures provenance and audit metadata are attached to modified elements, and writes version-controlled update records to preserve deterministic reproducibility of the graph state.

In some embodiments, the graph curation module 240 also supports session-specific querying and traversal operations used to access relevant portions of a patient’s longitudinal clinical memory graph. When a new therapy session begins, the graph curation module 240 may identify a starting node within the graph that most closely corresponds to recent psychological state variables. The graph curation module 240 may then traverse connected edges from this starting node, accessing temporal, associative, or causal links to locate adjacent nodes representing prior states, interventions, and outcomes. During traversal, the graph curation module 240 applies recency weighting and semantic relevance scoring to select nodes most pertinent to the present therapeutic context. From these selected nodes, the graph curation module 240 extracts psychological state information such as mood patterns, emotional stability indicators, thinking-pattern rigidity, attachment behaviors, therapeutic alliance measures, relapse-risk estimates, or some combination thereof. The resulting extracted data provide a structured, context-aware snapshot of longitudinal patient variables that can be passed to the prompt generation module 260 to inform model-driven intervention reasoning.

Following completion of a therapeutic session, the graph curation module 240 generates a dedicated session outcome node to record measured results associated with the intervention. This node may be populated with metadata and quantitative variables that include intervention effectiveness scores, patient response metrics, and broader therapeutic progress indicators derived from post-session multimodal analysis. The graph curation module 240 links this new outcome node to pre-existing psychological state nodes in the patient’s longitudinal clinical memory graph (LCMG) to maintain temporal continuity and reflect progression from the prior mental-health states to current measured outcomes. In addition, the module updates confidence scores and uncertainty measures for related psychological variables based on the quality and consistency of the observed response data. The updated state graph, comprising the new outcome node and recalibrated confidence values, is then persisted within the secure graph data store 230 for use in subsequent therapeutic reasoning cycles. The graph curation module 240 may follow a similar process to add and update other node types in the LCMG.

In some embodiments, linking the session outcome node (or another type of node) to existing psychological state nodes is performed using a temporal traversal algorithm executed by the graph curation module 240. The algorithm identifies psychological state nodes captured within a defined temporal window preceding the current session and establishes directed edges from those nodes to the newly created session outcome node to represent sequential therapeutic progression. Each edge is assigned temporal metadata specifying start and end timestamps corresponding to the related psychological states or interventions. The graph curation module 240 further updates edge-level confidence values to represent the statistical strength or reliability of continuity between antecedent states and the current outcome. Once updated, these modified edges are written back to the longitudinal graph with retained metadata that preserves chronological and causal relationships among patient states.

In addition to the outcome node example, the graph curation module 240 also manages other node categories within the longitudinal clinical memory graph (LCMG). When new multimodal data or clinician annotations are received, the graph curation module 240 may analyze the input context to determine whether adjustments should be made to existing traits, states, events, or interventions represented in the graph. For example, inferred or measured changes in mood, anxiety, or relational variables may trigger updates to dynamic state nodes, whereas stable psychological characteristics or personality indicators are maintained within trait prior nodes that may be updated more conservatively using slower temporal decay and higher persistence weighting.

The graph curation module 240 may also process situational or contextual triggers as event nodes. For example, when external stressors, social interactions, or contextual changes are identified from multimodal data streams or clinician input, the graph curation module 240 may create corresponding event nodes and attach metadata such as timestamps, activation levels, and provenance indicators. These events are then linked by causal or temporal edges to state nodes whose emotional or behavioral content exhibit measurable shifts following the event. This kind of graph update enables the system to encode observed cause-and-effect patterns between contextual triggers and psychological responses directly into the patient’s longitudinal data structure.

For sessions or exercises representing direct interventions, the graph curation module 240 may construct or update intervention nodes that record the therapeutic activity, duration, modality, and validation status from the clinician interface. These nodes are integrated with adjacent dynamic state and outcome nodes by intervention-response edges, capturing quantitative response measures such as effect magnitude, progress indices, and confidence metrics. Over time, accumulated intervention-response relationships may enable the analytics system 140 to compute causal effect sizes and learn which therapeutic approaches yield the most favorable outcomes for a particular patient.

Throughout this process, the graph curation module 240 may recalibrate edge weights, confidence values, and uncertainty bounds using probabilistic modeling and temporal decay functions to maintain coherent longitudinal trajectories within the LCMG. Each modification may be logged with provenance and version metadata to ensure that the graph remains auditable and reproducible. By managing node and edge creation in this structured manner, the graph curation module 240 preserves temporal continuity, captures evolving clinical meaning, and provides a persistent, interpretable foundation for adaptive therapy reasoning in subsequent sessions.

The integrated modalities datastore 250 stores therapeutic protocols, intervention templates, and modality-specific structures that the analytics system 140 may reference when proposing or evaluating candidate therapies. In various embodiments, the integrated modalities datastore 250 may include structured records for cognitive-behavioral frameworks, grounding or stabilization exercises, communication-coaching modules, values-clarification templates, habit-formation scaffolds, other evidence-based intervention information, or some combination thereof. Metadata may further include modality classification, contraindication markers, intensity parameters, jurisdictional availability tags, or some combination thereof. The integrated modalities datastore 250 provides standardized, safety-aligned therapeutic content that can be injected into prompt generation or intervention selection workflows.

The prompt generation module 260 generates candidate prompts for execution by generative or inference models hosted in the model-serving system 150. In one embodiment, the prompt generation module 260 receives inputs including information about the state graph for a patient as stored in the graph data store 230, extracted features from the multimodal inference system 220, relevant therapeutic templates retrieved from the integrated modalities datastore 250, or some combination thereof. Based on these elements, the prompt generation module 260 constructs structured prompts encoding patient context, longitudinal variables, and applicable modality logic for use by downstream generative models to propose candidate therapeutic interventions. Each generated prompt may include audit metadata, policy identifiers, and uncertainty indicators to support traceability and governed output generation.

In operation, the prompt generation module 260 may transmit the structured prompts to the model-serving system 150 for execution by one or more generative or inference models. The model-serving system 150 processes each prompt and returns generated responses, such as proposed therapeutic pathways, recommendations, or explanatory visualizations, in accordance with system policy constraints and model governance rules.

In some embodiments, the analytics system 140 may additionally include a deterministic constraint compiler that evaluates candidate therapeutic interventions prior to providing the candidate interventions to a user device. The constraint compiler maintains a machine-readable policy rule set that includes contraindication rules (e.g., prohibiting exposure-based interventions for patients with acute dissociative episodes), intensity thresholds (e.g., maximum escalation rates for graded activation protocols), and jurisdictional limitation policies (e.g., restricting certain psychedelic-assisted therapy preparation content to jurisdictions where such treatments are legally authorized). When a candidate intervention is received from the model serving system 150, the constraint compiler may parse the intervention into structured attributes including intervention type, intensity level, target psychological domain, and required clinical oversight level. Each attribute is matched against the policy rule set. If a violation is detected, the constraint compiler generates a gating decision: the intervention may be blocked entirely, modified to reduce intensity or scope, or flagged for mandatory clinician review before provision to a patient. The gating decision, its rationale, and the specific policy rules triggered may be logged as audit metadata and associated with the corresponding intervention node in the LCMG.

The returned responses are then provided to the user-interface module 270, which translates the model outputs into interactive interface components for presentation to patients or clinicians. The user-interface module 270 may adapt the generated content to reflect user role, confidence indicators, and compliance requirements.

### Longitudinal Clinical Memory Graph

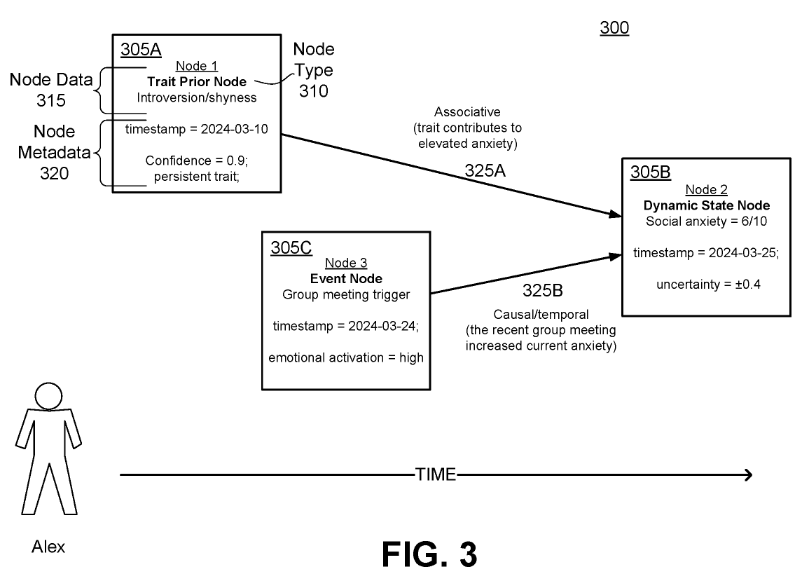

FIG. 3 illustrates an example Longitudinal Clinical Memory Graph (LCMG) 300 before a therapeutic intervention. The example depicted in FIG. 3 corresponds to a simplified patient scenario for a fictional patient, Alex, who experiences social anxiety. The example LCMG 300 visualizes how different pieces of therapeutic and contextual information are represented as interconnected data nodes and edges that evolve over time. The LCMG 300 includes three nodes 305 (specifically node 1 (305A), node 2 (305B), and node 3 (305C)). Each node 305 includes node data 315 describing information relevant to the patient’s psychological characteristics, current state, or contextual events. Each node 305 further includes a node type 310 describing the category of information represented in that node (for example, trait prior, dynamic state, or event). Additionally, each node 305 may include node metadata 320 such as timestamps, confidence values, and other parameters that enable longitudinal tracking and uncertainty handling within the patient’s record.

The nodes 305 in the example of FIG. 3 are connected by directed edges 325 (specifically edges 325A and 325B). Each edge 325 has a defined edge type that represents how one node influences another, such as associative, causal, or temporal. In this example, the nodes are arranged by the order of their timestamps to illustrate temporal progression. Node 1 (305A) and Node 3 (305C) both connect to Node 2 (305B), demonstrating how the patient’s underlying traits and recent experiences jointly contribute to the recorded dynamic mental state.

Node 1 (305A) is a trait prior node that records Alex’s introversion and shyness as an enduring personality characteristic. Node 1 (305A) includes metadata noting that the trait is persistent across sessions and is timestamped at 2024-03-10, with a confidence value of 0.9. Node 3 (305C) is an event node representing an external triggering event (specifically, a group meeting that elicited heightened stress). Node 3 (305C) includes node data 315 about the group meeting and metadata describing a high level of emotional activation during the event. Node 2 (305B) is a dynamic state node capturing Alex’s social anxiety level of 6/10 after the triggering event.

Edge 325A starts at node 1 (305A) and points to node 2 (305B). Edge 325A is classified as an associative edge, representing that Alex’s introversion trait contributes to the elevated social anxiety reflected in the dynamic state node. Edge 325B, starting at node 3 (305C) and pointing to node 2 (305B), is a causal/temporal edge indicating that the recent group meeting triggered or intensified the anxiety captured in node (305B). Collectively, the interconnected nodes and edges in FIG. 3 illustrate how the LCMG structures and preserves relational, temporal, and causal information about the patient’s mental-health trajectory across time, providing an interpretable foundation for adaptive digital therapy reasoning within the analytics environment

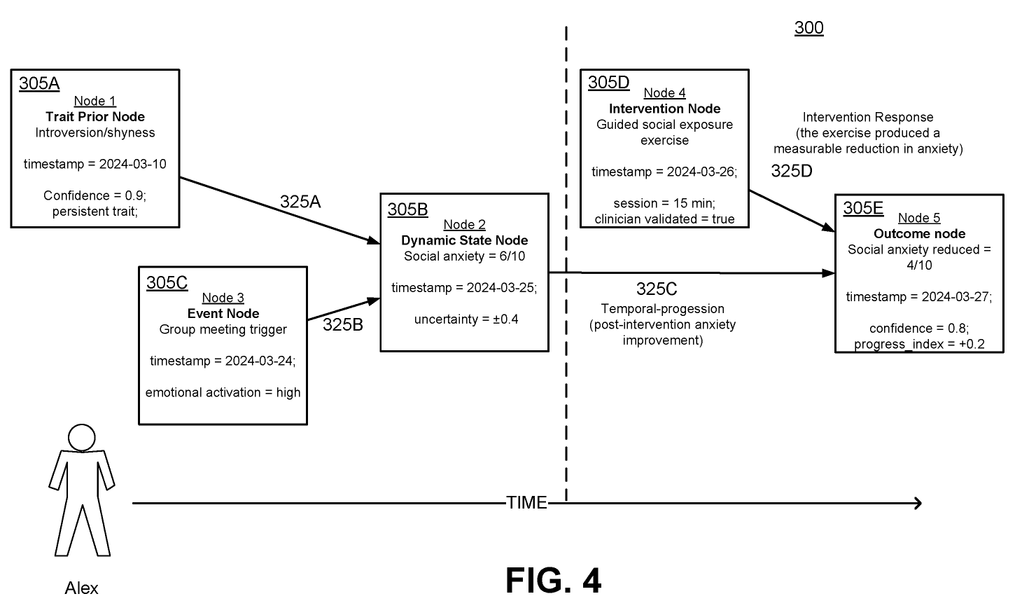

FIG. 4 illustrates an updated version of the Longitudinal Clinical Memory Graph (LCMG) 300 following a therapeutic intervention proposed and executed under the analytics system 140. In operation, the analytics system may use the graph depicted in FIG. 3 as structured input to a generative or inference model that processes the existing longitudinal data, including trait, dynamic state, and event information, to determine a suitable therapeutic intervention. Based on extracted features and graph context, the model may output a candidate therapy such as a guided social exposure exercise, which the user may complete with support from the patient interface and clinical oversight. After the session, the graph curation module updates the LCMG to reflect both the intervention and its measured outcome. The updates may be based on clinician inputs and additional multimodal inputs received from a patient device 110. The updates evolve the longitudinal record into the example configuration shown in FIG. 4.

The updated LCMG in FIG. 4 resembles the earlier graph in FIG. 3 but includes two new nodes 305D and 305E added to record the therapy session and its results. Node 4 (305D) is an intervention node that records a guided social exposure exercise completed by Alex with the system interface and clinician collaboration. The node data 315 for node 4 (305D) includes metadata 320 indicating a session duration of 15 minutesand a flag confirming that the intervention was clinician-validated. Node 5 (305E) is an outcome node that records that Alex’s social anxiety decreased to 4/10 following the intervention session. This node includes a timestamp indicating the time of measurement and a confidence value reflecting the reliability of the observed improvement.

Two directed edges 325C and 325D connect these new nodes to the existing graph, integrating the new information into the longitudinal model. Edge 325C begins at node 2 (305B) and points to node 5 (305E). Edge 325C is a temporal progression edge representing a transition from the previous dynamic state (social anxiety = 6/10) to the updated outcome state (social anxiety = 4/10), capturing measurable change to the patient condition over time. Edge 325D connects node 4 (305D) to node 5 (305E) and is classified as an intervention response edge indicating that the guided social exposure exercise directly contributed to the improvement recorded at the outcome node.

Collectively, the resulting LCMG 300 shown in FIG. 4 demonstrates how the analytics system records therapeutic interventions and corresponding outcomes within the longitudinal memory structure. Each new node and edge is time-indexed and includes appropriate metadata for provenance, confidence, and causal association, enabling interpretable tracking of therapeutic efficacy and progression within the patient’s digital clinical record.

### Updating and Using the LCMG

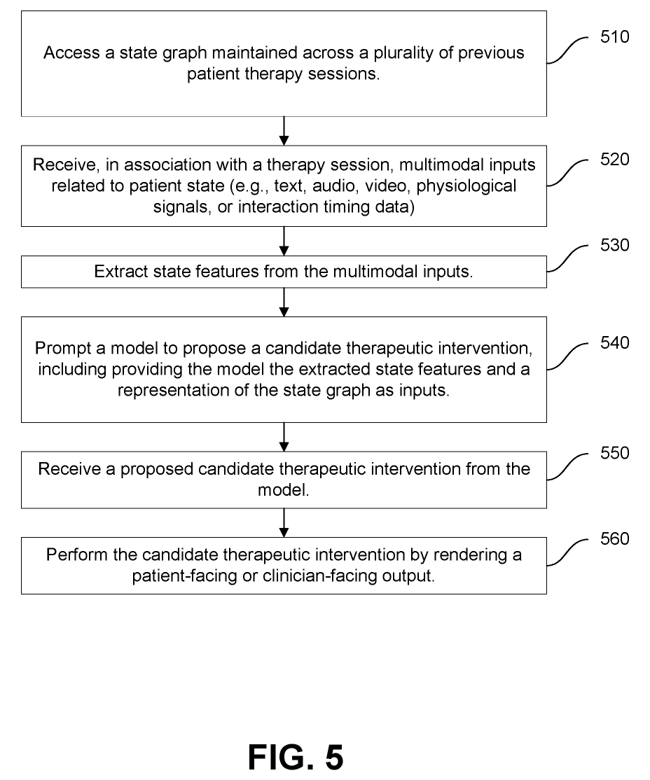

FIG. 5 is a flowchart depicting a process for using a patient LCMG to recommend a therapeutic intervention and updating the LCMG based on the outcome of the intervention, in accordance with an example embodiment.

The analytics system 140 accesses 510 a state graph maintained across a plurality of previous patient therapy sessions. This operation retrieves longitudinal data stored within the encrypted Longitudinal Clinical Memory Graph (LCMG) representing historical variables associated with the patient’s mental-health state and prior therapeutic interventions. The LCMG may include time-indexed nodes corresponding to trait priors, dynamic state variables, interventions, and outcomes, with associated confidence values and timestamps. By accessing these nodes and their relational edges, the analytics system 140 obtains a structured foundation reflecting how the patient’s psychological variables have evolved over time. Querying the graph may include traversing temporal, associative, causal edges, or some combination thereof to locate subgraphs corresponding to relevant recent sessions and extracting data useful for driving therapy decisions while preserving longitudinal continuity.

In some embodiments, to obtain a relevant subgraph, the analytics system 140 applies a query routine that filters nodes and edges based on contextual constraints such as recency of sessions, predefined therapeutic goals, or anomaly scores indicating deviations from baseline behavior. The query may utilize weighted traversal heuristics that prioritize edges with higher causal confidence or tighter temporal association, resulting in extraction of a subgraph centered on the most clinically salient episode sequence. In some embodiments, the analytics system 140 may additionally prompt a patient or clinician at a user device to add additional details related to the state graph.

The analytics system 140 receives 520 multimodal inputs related to patient state in association with a current therapy session. These inputs may include text, audio, video, physiological signal data, and interaction timing features collected through the patient interface or connected sensors. The multimodal data are synchronized across modalities and provide rich evidence streams describing observable indicators such as speech cadence, tone, facial expression changes, and affective timing patterns. The analytics system 140 uses secure communication protocols to receive this data and stores derived features within its inference layer for subsequent analysis, maintaining encryption boundaries and data integrity consistent with regulated deployment requirements.

The analytics system 140 extracts 530 state features from the multimodal inputs. Extracting state features may involve applying the multimodal spatiotemporal inference engines described previously (e.g., using temporal convolutional networks, sequence transformers, or vision transformers) to compute interpretable feature vectors capturing behavioral, emotional, and physiological markers. The extraction process converts unstructured data streams from text, audio, and video into normalized representations such as prosodic stress metrics, facial action unit dynamics, and gaze stability scores. Once extracted, these features are aligned temporally and written to time-indexed nodes in the LCMG as derived variables with confidence and uncertainty metadata, enabling probabilistic inference over time-evolving mental-health trajectories.

The analytics system 140 prompts 540 a model to propose a candidate therapeutic intervention. As part of the prompt, the analytics system may provide the model with the extracted state features and a relevant representation of the state graph. The system generates a structured input packet prompt that includes encoded contextual features from the multimodal inference layer and summarized graph variables such as recent anxiety measures, relational indicators, or prior interventions. The model processes this input using probabilistic reasoning and generates interpretable candidate interventions within safety boundaries established by supervisory policies, which may also be provided as part of the prompt or may be included in the model training. An example intervention may be recommending a guided social exposure exercise or a stabilizing reflection protocol based on observed trends in the patient’s state.

The analytics system 140 receives 550 the proposed candidate therapeutic intervention from the model. The received output may contain recommended intervention details, intensity settings, and rationale traces linking the inference to contributing state variables in the graph. The analytics system 140 evaluates the candidate against active safety constraints and clinical policies managed by its deterministic constraint compiler. Interventions violating contraindication or jurisdictional limitation rules may be blocked or modified prior to execution. Valid proposals are then passed downstream for rendering or further clinician validation. In some embodiments, interventions are provided to a clinician-facing client device (e.g., clinical client device 120) for approval or confirmation prior to presentation on an interface of a patient-facing client device (e.g., patient client device 110).

The analytics system 140 performs 560 the candidate therapeutic intervention by rendering either a patient-facing or clinician-facing output. For patient-facing contexts, this may involve delivering medication prompting, interactive exercises, grounding protocols, or supportive messages through the interface. In clinician-facing contexts, the analytics system 140 may produce dashboards, progress summaries, or structured documentation artifacts illustrating changes in patient state and intervention rationale. Once rendered, the system observes and records post-intervention changes in multimodal features, computes response metrics, and writes updated variables including new outcome nodes and causal edges back to the LCMG. The therapeutic actions and their outcomes are stored with timestamps and confidence values to extend longitudinal continuity of care and enable future sessions to build upon verified historical data for the patient.

The analytics system 140 observes changes to the multimodal inputs following the therapeutic intervention. After rendering the patient-facing or clinician-facing output, the system continues monitoring the patient’s subsequent text, audio, video, and other input streams to assess post-intervention effects. Variations in vocal tone, facial expression, body language, interaction timing, and physiological signals may be analyzed in conjunction with other patient inputs to determine whether the intervention produced measurable changes in emotional or behavioral state. The multimodal inference system computes deltas between pre- and post-intervention features, and updates confidence and uncertainty metrics to represent observed trends such as relaxation, stabilization, or residual distress.

The analytics system 140 updates the state graph based on the observed changes to the multimodal inputs. The graph curation module generates or modifies nodes and edges within the longitudinal clinical memory graph to reflect intervention outcomes. For example, a new outcome node may be created to capture session-specific response metrics, and temporal or causal edges are adjusted to connect the outcome with preceding dynamic state and intervention nodes. The update process may include propagating adjusted confidence weights across related nodes, refining uncertainty bounds, and recording provenance metadata detailing when and why specific updates occurred.

The analytics system 140 stores the updated state graph for use in subsequent therapy sessions. The revised LCMG, containing newly written nodes and recalibrated edges, is securely persisted within encrypted storage managed by the analytics environment. The system may encrypt the updated state graph and associate the graph with version identifiers supporting deterministic reconstruction and audit traceability. During later sessions, the analytics system 140 retrieves this stored graph to supply context and continuity for reasoning processes when making future therapeutic recommendations.

### Machine Learning Models

In various embodiments, a wide variety of machine learning techniques may be used. Examples include different forms of supervised learning, unsupervised learning, and semi-supervised learning such as decision trees, support vector machines (SVMs), regression, Bayesian networks, and genetic algorithms. Deep learning techniques such as neural networks, including convolutional neural networks (CNN), recurrent neural networks (RNN), long short-term memory networks (LSTM), transformers, linear recurrent neural networks such as Mamba, may also be used. For example, various multimodal feature extraction tasks performed by the multimodal inference system 220 and state-feature fusion tasks performed by the prompt generation module 260 may apply one or more machine learning and deep learning techniques. Additionally, models hosted by the model serving system 150 may use these techniques.

One or more deep neural networks (DNNs) may be used in various embodiments. A DNN may refer to an AI model that includes at least 5 hidden layers, at least 50 learnable computational nodes in the aggregate across the hidden layers, and at least 2,000 trainable parameters, which may include weights, biases, attention tensors, or other learned coefficients. In some embodiments, the DNN may include between 5 and 64 hidden layers or more. These embodiments encompass neural networks of varying sizes and complexities, including convolutional networks, recurrent networks, transformer-based architectures, and other variants that meet the minimum structural thresholds described above, as used by the model serving system 150 to process text, audio, video, or physiological inputs and to generate candidate therapeutic interventions.

In various embodiments, the training techniques for a machine learning model may be supervised, semi-supervised, or unsupervised. In supervised learning, models are trained on annotated mental-health records that pair multimodal signals (e.g., text, voice tone, facial expression, physiological data) with clinician-verified labels representing psychological state or therapeutic outcomes. For example, for a model trained to predict or classify risk level or emotional state, the training samples may be past session data with known outcomes. The labels for each training sample may include improved, unchanged, or deteriorated indicators of well-being. In some embodiments, labels may be multiclass, such as various domains of psychological state (mood, anxiety, engagement, etc.).

By way of example, the training set may include multiple past patient sessions captured through multimodal signals with known therapeutic outcomes. Each training sample in the training set corresponds to a previous therapy interaction, and the corresponding clinician-validated result serves as the label. Each sample may be represented as a feature vector including multiple dimensions such as linguistic content, prosody, facial affect intensity, gaze stability, interaction latency, and physiological metrics. Certain pre-processing techniques may normalize the values across feature dimensions to ensure comparability of multimodal data streams.

In some embodiments, an unsupervised learning technique may also be used. The training samples may be represented by feature vectors but may not be labeled. The analytics system may use clustering techniques to group sessions with similar behavioral dynamics, emotional trends, or therapeutic responses, thereby inferring latent psychological states without explicit clinical labeling. Semi-supervised training may combine both annotated and unannotated multimodal sessions to improve robustness.

A machine learning model may be associated with an objective function that generates a metric value describing the goal of training. For example, the objective may minimize the error between predicted and actual changes in symptom severity or emotional stability. In generative model contexts, the objective may include a cross-entropy or L2 loss that measures divergence between predicted and clinician-validated state transitions in the longitudinal clinical memory graph. The model may also incorporate auxiliary losses for prediction of engagement retention or risk-state estimation, balancing interpretability and accuracy.

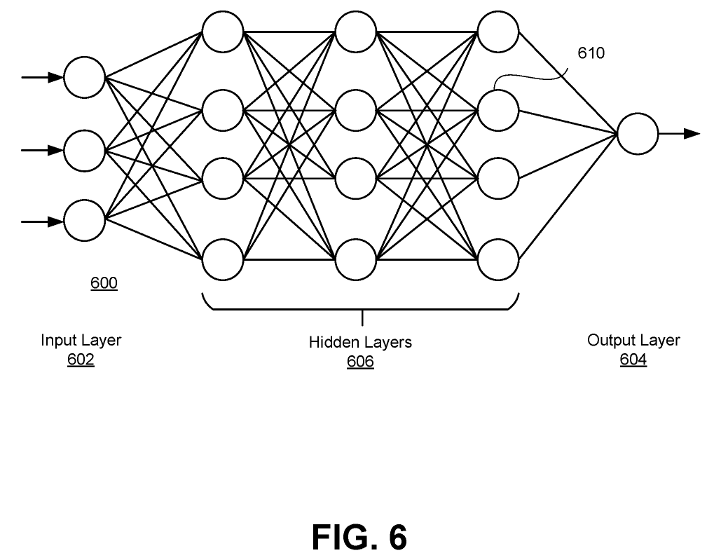

FIG. 6 illustrates a structure of an example neural network, in accordance with some embodiments. The neural network 600 may receive an input and generate an output. The input may be the multimodal feature vector derived from patient data (text, audio, video, physiological signals), and the output may be predictions of current state variables or proposals for therapeutic interventions. The network may include convolutional layers for processing visual data, transformer layers for text semantics, and recurrent sequences for temporal correlation across sessions.

The order and number of layers may vary by modality. Convolutional layers may be used for facial-expression detection; recurrent or transformer layers may model conversation dynamics and affective trajectories. Kernel sizes and attention heads may differ for processing fine-grained emotional cues versus longer temporal dependencies.

Training may include forward propagation and back-propagation across nodes associated with functions such as convolution, pooling, attention weighting, and activation (e.g., ReLU, tanh). Each node’s operation reflects transformations relevant to emotion recognition, language understanding, or physiological signal interpretation.

Training of a machine learning model may include iterative forward and backward passes using mental-health session data. For instance, a computing device may receive a training set of past multimodal sessions labeled with therapeutic outcomes. For each training sample, predicted emotional state or therapy effectiveness is generated and compared with clinician-verified labels. The system adjusts network weights through stochastic gradient descent to minimize the chosen loss function.

Each function in the neural network may include coefficients adjusted during training. Activation functions (ReLU, sigmoid, tanh) control nonlinear mapping of extracted features representing voice prosody or text sentiment. Performance is evaluated by comparing predictions (e.g., mood state change, engagement score) to ground-truth outcomes measured post-therapy.

Multiple training rounds may be performed until convergence, after which the trained model infers patient states or generates interventions during live sessions. The trained model predicts risk, engagement decay, or therapeutic response probability for decision support in ongoing care.

In some embodiments, the system periodically retrains the model on newly collected session data to improve accuracy and adapt to patient population drift. Retraining may occur as part of a continuous-learning cycle in which each verified intervention outcome updates the training corpus and fine-tunes the generative and inference models for better personalization and safety alignment.

In some embodiments, model distillation may transfer knowledge from large generative or multimodal reasoning models to smaller local models within the analytics system 140. For instance, a remote transformer-based generative model (teacher) may generate therapeutic recommendations, and a simplified local model (student) may learn from those outputs to operate on edge devices with reduced latency and footprint.

Feature-based distillation may align embeddings between the teacher model’s multimodal transformer and a student model, preserving latent affective and linguistic features while reducing computational cost. Hybrid approaches may combine response- and feature-level distillation to maintain therapeutic interpretability and efficiency.

This distillation process allows clinical AI deployments (e.g., on patient mobile apps or clinician dashboards) to achieve inference consistency while meeting regulatory and safety constraints, ensuring lower latency, privacy protection, and compliant operation in healthcare contexts.

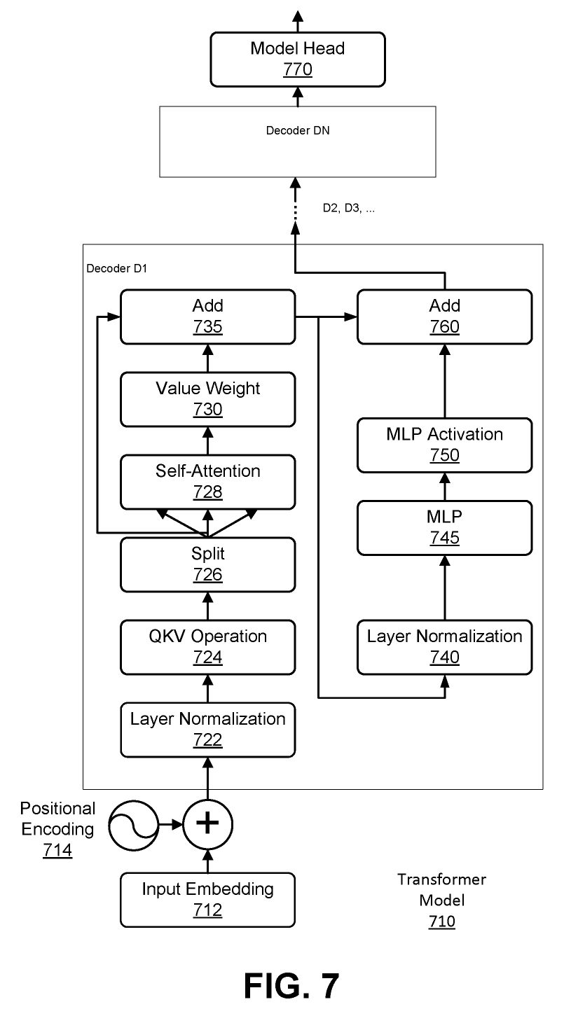

FIG. 7 is a conceptual diagram of functional blocks of a transformer-based neural network model 710, in accordance with some embodiments. For simplicity, the transformer-based neural network model 710 is referred to as a transformer model 710. The transformer model is an example of a machine-learning model discussed in this disclosure. An actual transformer model 710 may be a large language model that involves numerous neurons, such as a large number of decoders and parameters. The structure illustrated in FIG. 7 is part of a decoder for generating token attention. In a language-processing task related to therapeutic reasoning and intervention generation, the input may take the form of a sequence of words representing a structured prompt encoding multimodal state features and graph context. Each token represents a respective embedding in a latent space. Based on the input tokens, the transformer model 710 repeatedly generates a sequence of output tokens in an autoregressive manner that correspond to candidate therapeutic actions or interpretive rationales for clinical prompts.

In some embodiments, a transformer model 710 includes a set of N decoders, D1, D2, … DN. Each decoder receives input representations and generates output representations. For example, the first decoder D1 generates intermediate embeddings contextualized for patient psychological state variables and multimodal cues. Each subsequent decoder refines these embeddings using prior decoder outputs and the state-graph context until a final therapeutic recommendation vector is produced. Some decoders may correspond to analyzing data dimensions that model text, audio, video, and physiological indicators. These multimodal streams are used to perform feature integration and therapeutic inference, enabling the model to reason across linguistic, affective, and behavioral data.

The transformer model 710 may include a model head block 770 that receives the set of output representations from the final decoder DN and generates an output token as the output for the current iteration. This output may represent natural-language therapeutic guidance, clinician-facing documentation text, or intervention rationale according to safety and regulatory policies managed by the analytics system.

As shown in FIG. 7, a decoder in the transformer model 710 includes a first layer-normalization block 722, a query-key-value (QKV) operation block 724, a split block 726, a self-attention block 728, a value-weight block 730, a first add block 735, a second layer-normalization block 740, multi-layer perceptron (MLP) block 745, an MLP activation block 750, and a second add block 760. The operations in the first decoder D1 are exemplary; subsequent decoders may include similar operations. These layers allow the model to attend dynamically to relevant features in multimodal inputs and to latent state-graph variables describing the patient’s historical therapeutic context.

FIG. 7 illustrates a flow for the attention mechanism of a transformer model 710. The transformer model 710 receives an input sequence such as encoded state-graph data and multimodal embeddings collected from patient and clinician-facing interfaces. Each symbol is converted into a token that takes the form of an embedding vector. The sequence of symbols is represented as a matrix of embedding vectors, each embedding arranged in a row of the matrix. The layer-normalization block 722 receives the matrix and normalizes its values to stabilize input variance across sessions and modalities.

During training, the transformer model 710 may be trained in an autoregressive manner using masked label prediction. The input may be a therapeutic prompt sequence encoding prior session data and partially masked outcome labels. To simulate intervention prediction, the system applies masking where unknown intervention types or outcomes are hidden. The decoder attends only to previously observed state nodes and validated interventions while predicting masked positions corresponding to outcome nodes. The objective minimizes prediction error between masked positions and true therapeutic identifiers, enabling the transformer to model long-range dependencies and infer causal relationships between patient states and therapy outcomes.

The QKV operation block 724 receives the normalized dataset and performs projections to generate query, key, and value matrices. The operation applies learned weights to align representations with contextual signals derived from the longitudinal clinical memory graph (LCMG). The QKV operation models relationships among psychological variables, intervention history, and multimodal affect markers to produce attention distributions guiding therapeutic reasoning and content generation.

The split block 726 splits the QKV output into query, key, and value matrices. The self-attention block 728 uses these matrices to generate an attention matrix, applying softmax scaling. The softmax converts logit scores into attention probabilities indicating relevance between patient states and proposed interventions. This attention function allows the transformer model 710 to associate multimodal patterns with therapy outcomes and prioritize nodes with higher causal relevance within the longitudinal graph.

The value-weight block 730 receives the attention-score data to generate an attention dataset representing weighted combinations of value vectors. The results are concatenated in the add block 735 and further normalized by 740. These operations refine the interpretive context and produce latent embeddings that encode therapeutic rationale, patient progress indicators, and confidence metrics for graph updates.

Each decoder may include one or more MLP blocks 745 and MLP activation blocks 750 configured with nonlinear activation functions. The activation functions introduce non-linearity and support mapping of complex psychological transitions. Common activations may include ReLU, tanh, sigmoid, or GeLU. The MLP layers perform feature extraction across multimodal signals, generate compact context embeddings, and select token sequences for subsequent decoding related to therapy planning. Outputs are concatenated by 760 to complete the cross-modal fusion pipeline.

The output of the first decoder D1 is passed to subsequent decoders until final output data are generated. Each decoder may operate with different trained parameters focused on progressively refined aspects of patient mental-state modeling or intervention inference. The model head block 770 receives the final output from DN and determines an output token that forms natural-language recommendations or log entries. A softmax operation performed at the LM head selects the next token, resulting in governed therapeutic language or clinician summary text integrated into longitudinal documentation of patient progress.

### Additional Considerations

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more sub-processing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

The description herein may describe processes and systems that use machine-learning models in the performance of their described functionalities. A “machine-learning model,” as used herein, comprises one or more machine-learning models that perform the described functionality. Machine-learning models may be stored on one or more computer-readable media with a set of weights. These weights are parameters used by the machine-learning model to transform input data received by the model into output data. The weights may be generated through a training process, whereby the machine-learning model is trained based on a set of training examples and labels associated with the training examples. The training process may include: applying the machine-learning model to a training example, comparing an output of the machine-learning model to the label associated with the training example, and updating weights associated for the machine-learning model through a back-propagation process. The weights may be stored on one or more computer-readable media, and are used by a system when applying the machine-learning model to new data.

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

What is claimed is:

1. A computer-implemented method comprising:
accessing a state graph maintained across a plurality of previous patient therapy sessions, wherein the state graph comprises time-indexed nodes representing psychological state variables and therapeutic intervention history associated with a patient;
in association with a current therapy session, receiving multimodal inputs related to patient state including at least two of text, audio, video, physiological signals, or interaction timing data;
extracting state features from the multimodal inputs;
prompting a generative language model to propose a candidate therapeutic intervention, wherein the prompting comprises providing to the generative language model the extracted state features and a representation of the state graph;
receiving a proposed candidate therapeutic intervention from the generative language model; and
performing the candidate therapeutic intervention by rendering a patient-facing output.

2. The method of claim 1, further comprising:
observing changes to the multimodal inputs following the therapeutic intervention;
updating the state graph based on the observed changes to the multimodal inputs; and
storing the updated state graph for use in subsequent therapy sessions.

3. The method of claim 2, wherein updating the state graph comprises:
creating a session outcome node following completion of the therapeutic session;
populating the session outcome node with intervention effectiveness scores, patient response metrics, and therapeutic progress indicators;
linking the session outcome node to existing psychological state nodes in the state graph based on temporal progression;
updating confidence scores for related psychological state variables based on the observed changes to the multimodal inputs; and
storing the updated state graph with the session outcome node for use in the subsequent therapy sessions.

4. The method of claim 3, wherein linking the session outcome node to the existing psychological state nodes in the state graph based on the temporal progression comprises:
identifying psychological state nodes recorded within a defined temporal window preceding the current session;
establishing directed edges from the identified psychological state nodes to the session outcome node;
assigning temporal metadata related to start and end times of the related psychological states to each of the established directed edges;
updating confidence values associated with the established directed edges; and
storing the established directed edges in the state graph.

5. The method of claim 1, wherein extracting features from the multimodal inputs comprises extracting facial expression changes from video signals.

6. The method of claim 1, wherein extracting features from the multimodal inputs comprises extracting word patterns from text inputs.

7. The method of claim 1, further comprising performing the candidate therapeutic intervention by rendering a clinician facing output.

8. The method of claim 7, wherein the clinician facing output comprises at least one of longitudinal dashboards displaying schema rigidity trends, attachment activation cycles, alliance durability metrics, relapse probability curves, dropout hazard estimates, belief updating trajectories, structured documentation artifacts, care plan updates, or some combination thereof, and wherein the clinician facing output enables clinician intervention by providing recommendations for clinical oversight and treatment planning.

9. The method of claim 1, wherein accessing the state graph comprises:
querying the state graph using a current session context to locate a starting node;
traversing edges from the starting node to related nodes;
selecting nodes based on recency weights and relevance to current therapeutic context; and
extracting psychological state information from the selected nodes.

10. The method of claim 9, wherein extracting the psychological state information from the selected nodes includes extracting at least one of: mood patterns, emotional stability measures, thinking pattern rigidity, attachment behaviors, therapeutic relationship strength, and relapse risk estimates.

11. The method of claim 1, wherein the patient-facing output comprises at least one of: cognitive restructuring prompts, skills coaching protocols, stabilization exercises, between-session coursework, values reflection support, sleep and circadian supports, communication coaching, relational skill exercises, habit formation scaffolding, and relapse prevention planning.

12. The method of claim 1, wherein nodes of the state graph represent at least one of: trait prior variables, dynamic mental healthcare state variables, intervention events, and outcome variables.

13. The method of claim 1 wherein edges of the state graph represent at least one of: temporal relationships, associative relationships, intervention-response relationships, and causal relationships between the nodes.

14. A non-transitory computer-readable medium storing instructions that, when executed by one or more processors, cause the one or more processors to:
access a state graph maintained across a plurality of previous patient therapy sessions, wherein the state graph comprises time-indexed nodes representing psychological state variables and therapeutic intervention history associated with a patient;
in association with a current therapy session, receive multimodal inputs related to patient state including at least two of text, audio, video, physiological signals, or interaction timing data; extract state features from the multimodal inputs;
prompt a generative language model to propose a candidate therapeutic intervention, wherein the prompting comprises providing to the generative language model the extracted state features and representation of the state graph;
receive a proposed candidate therapeutic intervention from the generative language model; and
perform the candidate therapeutic intervention by rendering a patient-facing output.

15. The non-transitory computer-readable medium of claim 14, wherein the instructions further cause the one or more processors to:
observe changes to the multimodal inputs following the therapeutic intervention;
update the state graph based on the observed changes to the multimodal inputs; and
store the updated state graph for use in subsequent therapy sessions.

16. The non-transitory computer-readable medium of claim 15, wherein the instructions for updating the state graph further cause the one or more processors to:
create a session outcome node following completion of the therapeutic session;
populate the session outcome node with intervention effectiveness scores, patient response metrics, and therapeutic progress indicators;
link the session outcome node to existing psychological state nodes in the state graph based on temporal progression;
update confidence scores for related psychological state variables based on session outcome data; and
store the updated state graph with the session outcome node for use in the subsequent therapy sessions.

17. The non-transitory computer-readable medium of claim 16, wherein linking the session outcome node to the existing psychological state nodes in the state graph based on the temporal progression further causes the one or more processors to:
identify psychological state nodes recorded within a defined temporal window preceding the current session;
establish directed edges from the identified psychological state nodes to the session outcome node;
assign temporal metadata related to start and end times of the related psychological states to each of the established directed edges;
update confidence values associated with the established directed edges; and
store the established directed edges in the state graph.

18. The non-transitory computer-readable medium of claim 14, wherein the instructions for extracting features from the multimodal inputs further cause the one or more processors to extract facial expression changes from video signals.

19. The non-transitory computer-readable medium of claim 14, wherein the instructions for extracting features from the multimodal inputs further cause the one or more processors to extract word patterns from text inputs.

20. A system comprising:
one or more processors; and
memory configured to store instructions that, when executed by the one or more processors, cause the one or more processors to:
access a state graph maintained across a plurality of previous patient therapy sessions, wherein the state graph comprises time-indexed nodes representing psychological state variables and therapeutic intervention history associated with a patient;
in association with a current therapy session, receive multimodal inputs related to patient state including at least two of text, audio, video, physiological signals, or interaction timing data;
extract state features from the multimodal inputs;
prompt a generative language model to propose a candidate therapeutic intervention, wherein the prompting comprises providing to the generative language model the extracted state features and a representation of the state graph;
receive a proposed candidate therapeutic intervention from the generative language model; and
perform the candidate therapeutic intervention by rendering a patient-facing output.
